(19) United States
(12) Patent Application Publication
(10) Pub. No.: US 2013/0309732 A1
(43) Pub. Date: Nov. 21, 2013

(54) BIOSYNTHESIS METHODS OF NOREPHEDRINE WITH SPECIFIC OPTICAL ACTIVITIES

(71) Applicants: CHAO-HUNG KAO, TAIPEI CITY (TW); JIAN-SIN LIN, MIAOLI COUNTY (TW)

(72) Inventors: CHAO-HUNG KAO, TAIPEI CITY (TW); JIAN-SIN LIN, MIAOLI COUNTY (TW)

(73) Assignee: HUNGKUANG UNIVERSITY, TAICHUNG CITY (TW)

(21) Appl. No.: 13/475,007

(22) Filed: May 18, 2012

(51) Int. Cl. C12P 13/00

(52) U.S. Cl. 435/128

(57) ABSTRACT

A biosynthesis method of norephedrine with specific optical activities is revealed to convert and generate optical isomers with specific optical activities by biocatalysis. A two-step biotransformation reaction is carried by a whole-cell biocatalyst for converting reaction substrates, benzaldehyde and pyruvate, to L-phenylacetylcarbinol (L-PAC) in the first step and the yield of the L-PAC is 99%, and then an amino donor (L-alanine) is added and the transamination of the L-PAC is catalyzed by a transaminase with optical specificity for biosynthesizing the norephedrine with high optical purity. The pyruvate is produced from the amino donor, L-alanine, by the transamination in the reaction system, so that the pyruvate is regenerated in the reaction system without being added again.

### BACKGROUND OF THE INVENTION

1. Field of the Invention

The present invention relates a biosynthesis method of norephedrine with specific optical activities. More particularly, a creative designed method for biosynthesis of norephedrine with specific optical activities is unnecessarily to further separate and purify optical isomers from the reaction products. The formation of byproducts is also decreased. Furthermore, pyruvate is produced from the amino donor, L-alanine, by transamination in the reaction system, so that the pyruvate is regenerated in the reaction system without being added again to greatly reduce the reaction cost and raise the reaction efficiency.

2. Description of Related Art

Norephedrine is a kind of natural alkaloids and mainly exists in the Khat (Catha edulis, Arabian tea) and the Ephedraceae plants. The optical isomers of the norephedrine could be as starting materials for synthesizing many chiral compounds, such as chiral oxazoline, pipridines, aziridines, imidazolines, etc. The foregoing chiral compounds are very important materials for the application of organic synthesis. It is more important that the norephedrine with specific optical activity and its derivatives could be as chiral auxiliaries, chiral ligands or chiral catalysts in asymmetric reactions. The asymmetric reactions include enolate alkylation, aldol reaction, α or β-amino acid synthesis, epoxides to allylic alcohol reaction, oxazaborolidine reaction, hydrogen transfer reaction and alkylation to aldehydes reaction. Therefore, the norephedrine with specific optical activity and its derivatives occupy an important place in the application of the asymmetric reactions.

The norephedrine is synthesized mainly by the two following method:

The first synthetic method is chemical synthesis, which performs hydrogenation by utilizing metal-reducing agent or chemical catalyst to produce reaction products. The chemical synthesis has advantages as the cheap material used and a high yield of the product. However, the final product of the chemical synthesis is racemic mixture; the process for separating specific optical product from the racemic mixture is very complicated, time-consuming, high cost, and less efficiency. Moreover, the separating process also requires a lot of organic agents which causes environment pollution and is not cost-effective.

The second synthetic method is fermentation-chemical synthesis. The material of the fermentation-chemical synthesis is L-phenylacetylcarbinol (L-PAC) produced from yeast fermentation. The process of the fermentation-chemical synthesis performs reduction reaction by utilizing metal-reducing agent, and then performs extracting and drying steps to give products. The best advantage of the fermentation-chemical synthesis is that all carbonyl groups bonding to chloride of the L-phenylacetylcarbinol are R form so that one of stereoselectivity step could be omitted. The products of the fermentation-chemical synthesis are only two kinds of isomers so that the process for separating isomers could be simplified. The reactants, L-phenylacetylcarbinol, could be given by yeast fermentation. In other words, the L-phenylacetylcarbinol is biosynthesized by yeast oxidoreductases. However, the biosynthesis of the L-phenylacetylcarbinol requires pyruvate as substrate, the cost of which is high. Moreover, the benzaldehyde may be oxidized to benzoic acid or may be reduced to benzyl alcohol by alcohol dehydrogenase in yeast. The L-phenylacetylcarbinol may also be reduced to 2-Phenyl-1, 3-propanediol (PAC-diol). The production of the foregoing byproducts exhausts the substrates to cause that the yield of the L-phenylacetylcarbinol is decreased and that the reaction products should be separated and purified by a complicated process. Although it is more efficient to produce purified optical isomer by synthesis method by utilizing the L-phenylacetylcarbinol as material, the cost of the material is higher and the reaction also requires organic reagents. Furthermore, the metal catalysis is exothermic reaction and the hydrogenation occurs under high pressure. The metal catalysis and the hydrogenation are highly dangerous.

### SUMMARY OF THE INVENTION

Therefore it is a primary object of the present invention to provide a biosynthesis method of norephedrine with specific optical activities in order to achieve the purpose of better practical value and the above object.

The biosynthesis method of norephedrine with specific optical activities in the present invention mainly converts and generates optical isomers with specific optical activities by biocatalysis. A two-step biotransformation reaction is carried by a whole-cell biocatalyst for converting reaction substrates, benzaldehyde and pyruvate, to L-phenylacetylcarbinol (L-PAC) in the first step and the yield of the L-PAC is 99%, and then an amino donor (L-alanine) is added and the transamination of the L-phenylacetylcarbinol is catalyzed by a transaminase with optical specificity for biosynthesizing the norephedrine with highly optical purity; thereby it is unnecessary to further separate and purify the optical isomers from the reaction products. The formation of the byproducts is also decreased. Furthermore, the pyruvate is produced from the amino donor, L-alanine, by transamination in the reaction system, so that the pyruvate is regenerated in the reaction system without being added again to greatly reduce the reaction cost and raise the reaction efficiency.

### BRIEF DESCRIPTION OF THE DRAWINGS

The techniques, objects, and effects of the present invention can be more fully understood by reading the following detailed description of the embodiment, with reference made to the accompanying drawings, wherein

FIG. 1 is an illustration of a chemical reaction of the present invention;

FIG. 2 is a diagram of the analysis result of the norephedrine concentration which is biosynthesized from the L-phenylacetylcarbinol by recombinant E. coli containing transaminase gene of the present invention;

FIG. 3 is a diagram of optical activity analysis of the (1R, 2S)-norephedrine standard and the (1S, 2R)-norepherine standard of the present invention.

FIG. 4 is a diagram of optical activity analysis of the (1S, 2R)-norepherine standard of the present invention.

FIG. 5 is a diagram of optical activity analysis of the (1R, 2S)-norephedrine standard of the present invention.

FIG. 6 is a diagram of optical activity analysis for the norephedrine biosynthesized by transformed E. coli, of the present invention.

FIG. 7 is a diagram of ion spectra of the norephedrine standard of the present invention.

FIG. 8 is a diagram of ion spectra of the decomposed norephedrine standard of the present invention.

FIG. 9 is a mass chromatogram of the norephedrine standard of the present invention.

FIG. 10 is a mass chromatogram of the present invention for the norephedrine biosynthesized by E. coli (pQE-CvTA).

FIG. 11 is an illustration of the present invention for a chemical reaction of biosynthesizing norephedrine from catalysis of transaminase (aminotransferase) and acetohydroxyacid synthase pair to the reaction between benzylamine and pyruvate.

### DETAILED DESCRIPTION OF THE PREFERRED EMBODIMENT

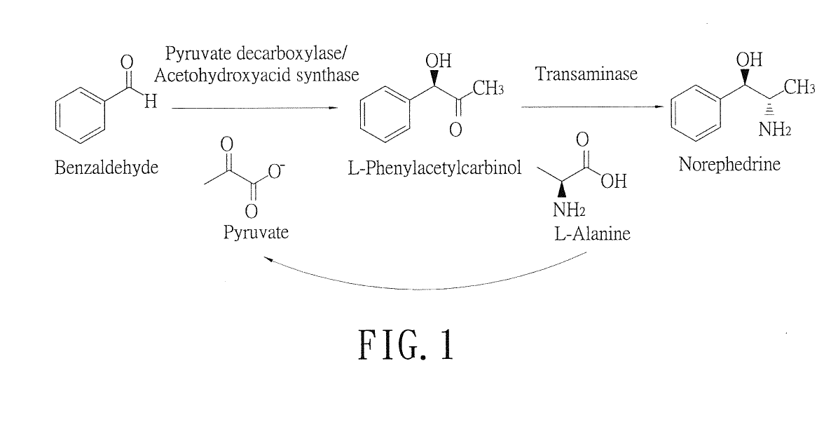

Refer to FIG. 1, it is an illustration of a chemical reaction of the present invention. The norephedrine with specific optical activities is mainly biosynthesized by biocatalysis in the present invention. First, the substrates, benzaldehyde and pyruvate, are catalyzed by acetohydroxyacid synthase, acetolactate synthase, or pyruvate decarboxylase for biosynthesizing L-phenylacetylcarbinol (L-PAC), and then the last carbon atom of carbonyl group on the side chain of the L-phenylacetylcarbinol is substituted for the amino group of amino donor catalyzed by a transaminase to convert the L-phenylacetylcarbinol to norephedrine, thereby accomplishing a two-step biosynthesis of the norephedrine. The origin of the foregoing acetohydroxyacid synthase is a bacterial strain with the acetohydroxyacid synthase activity, a transformed strain with the acetohydroxyacid synthase activity, or a purified enzyme with the acetohydroxyacid synthase activity, and the origin of the acetohydroxyacid synthase gene is salmonella typhimurium or Escherichia coli. The origin of the foregoing pyruvate decarboxylase is a bacterial strain with the pyruvate decarboxylase activity, a transformed strain with the pyruvate decarboxylase activity, or a purified enzyme with the pyruvate decarboxylase activity, and the origin of the pyruvate decarboxylase gene is Saccharomyces species, Hanseniaspora uvarum, Kluyveromyces species, Aspergillus species, Zymomonas mobilis, Sarcina ventriculi, Acetobacter specie, Erwinia amylovora, Schizosaccharomyces pombe, Candida utilis, Candida tropicalis, or Candida albicans. The origin of the foregoing transaminase is a bacterial strain with the transaminase activity, a transformed strain with the transaminase activity, or a purified enzyme with the transaminase activity. The foregoing transaminase can be β-alanine:pyruvate transaminases, omega transaminases, omega-amino acid:pyruvate transaminases, beta-alanine-alpha-alanine transaminases, amine:pyruvate transaminase.

Therefore, the present invention could perform the following embodiments:

### Embodiment 1 Construction of the Expression Vector

1. Construction of the Acetohydroxyacid Synthase (AHAS) Gene Expression Vector:

PCR primers for gene cloning were designed according to the AHAS gene sequence (NCBI CP001665 and NC_{—}012947) of E. coli BL21 (DE3). NCBI CP001665 and NC_{—}012947 were the gene sequence of ilvB and ilvN, respectively. Because the ilvB and ilvN gene formed an operon in E. coli and the gap between the open reading frame of these two structure genes were only 3 bases, according to the start site and end site sequence of the open reading frame of the ilvBN operon gene, the 5′ primer and 3′ primer for PCR amplifying ilvBN gene operon fragment from E. coli BL21 (DE3) chromosome were designed first. The fragment size of the ilvBN gene was about 2 kb. After digesting by restriction enzyme, the ilvBN gene fragment was cloned into an expression vector pQE-30 which containing T5 promoter and lac operator to form a pQE-AHAS I plasmid. The pQE-AHAS I plasmid was transformed into E. coli. The successfully transformed E. coli colony was picked up and induced gene expression by Isopropyl β-D-1-thiogalactopyranoside (IPTG). The protein expression level was analyzed by SDS-PAGE.

2. Construction of the Transaminase (TA) Gene Expression Vector:

PCR primers for gene cloning were designed according to the transaminase gene sequence of Chromobacterium violaceum (NCBI NP_{—}901695). The transaminase gene fragment was amplified by PCR from Chromobacterium violaceum chromosome. The transaminase gene fragment size was about 1.4 kb. After digesting by restriction enzyme, the transaminase gene fragment was cloned into an expression vector pQE-30 which containing T5 promoter and lac operator to form a pQE-CvTA plasmid. The pQE-CvTA plasmid was transformed into E. coli. The successfully transformed E. coli colony was picked up and induced gene expression by Isopropyl β-D-1-thiogalactopyranoside (IPTG). The protein expression level was analyzed by SDS-PAGE.

3. Construction of Co-Expression Vector and Expression of AHAS and CvTA:

Using the foregoing pQE-CvTA plasmid as template, the CvTA gene was amplified by PCR. The amplified PCR product contained T5 promoter, lac operator, and 6×His-tag sequence at 5′ end and λ transcription terminator at 3′ end. The PCR product was collected and ligated with the pQE-AHAS I plasmid which was digested by restriction enzyme before to form a pQE-CoTA plasmid, and the pQE-CoTA plasmid was transformed into E. coli. The pQE-CoTA plasmid could co-express AHAS and CvTA gene in the single E. coli host cell under being induced by individual T5 promoter.

### Embodiment 2

A two-step process for biosynthesizing norephedrine by acetohydroxyacid synthase transformed strain and transaminase transformed strain:

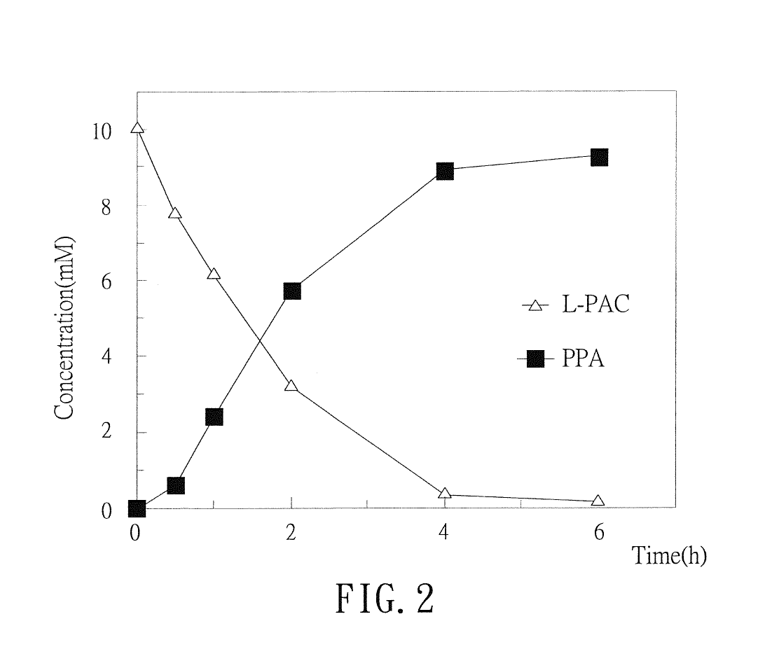

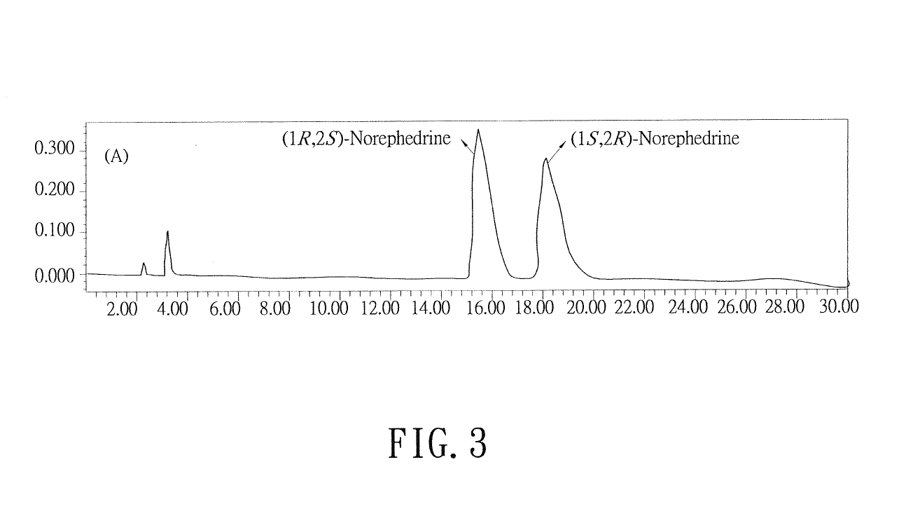

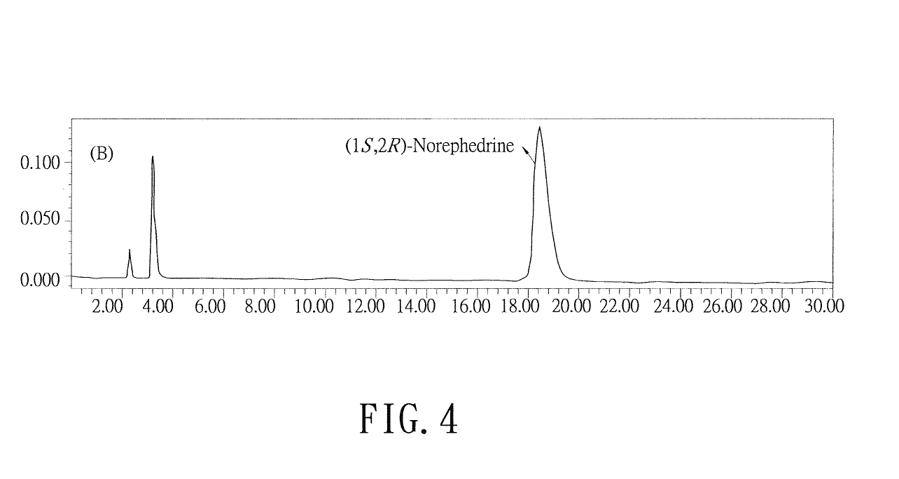

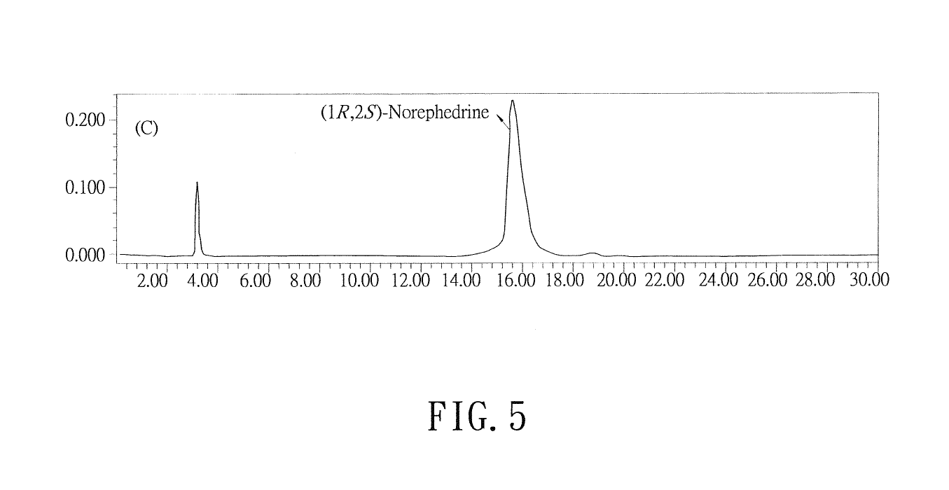

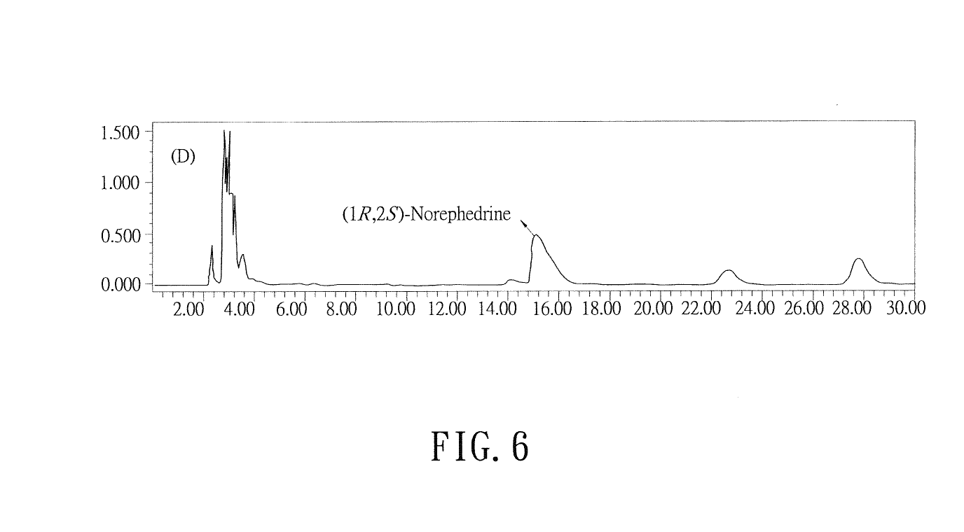

The transformed E. coli colony containing pQE-AHAS I plasmid was picked up and incubated in the LB broth (containing 50 mg/ml ampicillin) at 37° C. until the OD 600 of the broth culture was more than 0.8. Then, Isopropyl β-D-1-thiogalactopyranoside (IPTG) was added into the broth culture in which the IPTG final concentration was 1 mM, and the broth culture was shaken and incubated at 28° C. for 4 hours to induce gene expression. The induced broth culture was centrifuged by 6000 rpm for 15 minutes at 4° C. After centrifuging, the packed bacterial cells were collected and washed by 100 mM sodium phosphate buffer (pH 7.0) and centrifuged under the same condition again. After the second centrifuging, the supernatant was discarded, and the wet weight of the bacterial cells was measure. One gram (wet weight) of the bacterial cells was suspended in the sodium phosphate buffer (pH 7.0) and incubated at 37° C. for 12 hours for after use.
1. Step 1: First, L-phenylacetylcarbinol was biosynthesized through a method catalyzed by acetohydroxyacid synthase. The bacterial solution with pretreatment was centrifuged by 6000 rpm for 15 minutes at 4° C. After centrifuging, the supernatant was discarded, and 40 ml reaction solution was added into the packed cells to incubate for 12 hours at 37° C. The reaction solution contained 40 mM benzaldehyde, 40 mM pyruvic acid, 0.5 mM Dithiothreitol (DTT), 60 mM potassium chloride (KCl), 0.1 mM Thiamine (DhTP), 5 mM Magnesium chloride (MgCl₂.6H₂O) and 100 mM sodium phosphate buffer (pH 7.0). After incubation, the reaction solution was collected and analyzed by High-performance liquid chromatography (HPLC). The condition for HPLC analysis was as following: the mobile phase was 30% acetonitrile, the column was a C18 column, the flow rate was 1.5 ml/min, and the sample was detected at a wavelength of 283 nm. The retention time of the product, L-phenylacetylcarbinol, and the substrate, benzaldehyde, was 6.3 minute and 10.7 minute, respectively. According to the analysis result, the yield of L-phenylacetylcarbinol was 99%.
2. Step 2: A 100 mM sodium phosphate buffer (pH 7.0) was as a base to prepare a reaction solution containing 10 mM L-phenylacetylcarbinol (L-PAC), 100 mM L-alanine and 1 mM pyridoxal phosphate (PLP) as co-enzyme. The reaction solution was added into the tube containing IPTG-induced E. coli (pQE-CvTA) cells (wet weight: 0.2 g) to mix well and incubated at 37° C. for 0-6 hours. The induced E. coli cells had transaminase activity to perform transamination for converting the L-PAC to the norephedrine. The reaction products were collected and analyzed by HPLC and LC/MS/MS. The condition for HPLC analysis was as following: the mobile phase was 13% acetonitrile which was adjusted to pH 2 by perchloric acid, the column was a C18 column, the flow rate was 1.5 ml/min and the sample was detected at a wavelength of 210 nm. The retention time of the norephedrine and the L-phenylacetylcarbinol was 7.78 minute and 19.87 minute, respectively. Please refer to FIG. 2. FIG. 2 is a diagram of the analysis result of the norephedrine concentration which is biosynthesized from the L-phenylacetylcarbinol by recombinant E. coli containing transaminase gene of the present invention. According to the analysis result, the concentration of the norephedrine was 9.2 mM, and the yield of the norephedrine was 92%. Furthermore, the optical property of the norephedrine was analyzed by Chiral-HPLC. The condition for Chiral-HPLC analysis was as following: the mobile phase was perchoric acid (pH 2.0), the column was a Crownpak CR(+) column, the flow rate was 0.5 ml/min, and the sample was detected at a wavelength of 205 nm. The retention time of the norephedrine was 16.09 minute. Please refer to FIG. 3 to FIG. 6. FIG. 3 is a diagram of optical activity analysis of the (1R, 2S)-norephedrine standard and the (1S, 2R)-norepherine standard. FIG. 4 is a diagram of optical activity analysis of the (1S, 2R)-norepherine standard. FIG. 5 is a diagram of optical activity analysis of the (1R, 2S)-norephedrine standard. FIG. 6 is a diagram of optical activity analysis of the norephedrine biosynthesized by transformed E. coli. The retention time and the pattern of absorption spectrometry of the final product were consistent with that of the (1R, 2S)-norephedrine standard. Therefore, the optical property of the final product was confirmed to be (1R, 2S)-norephedrine.

### Embodiment 3

Structure Identification of the Reaction Product by LC/MS/MS Analysis:

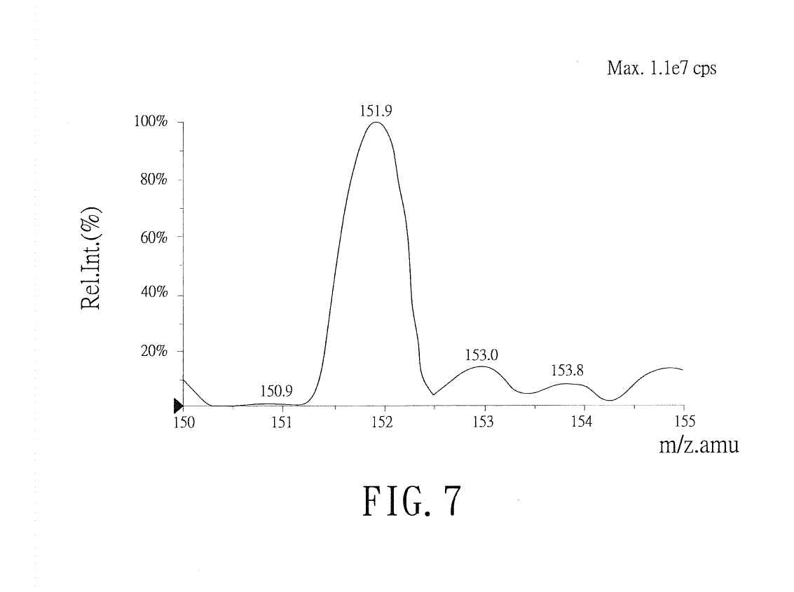

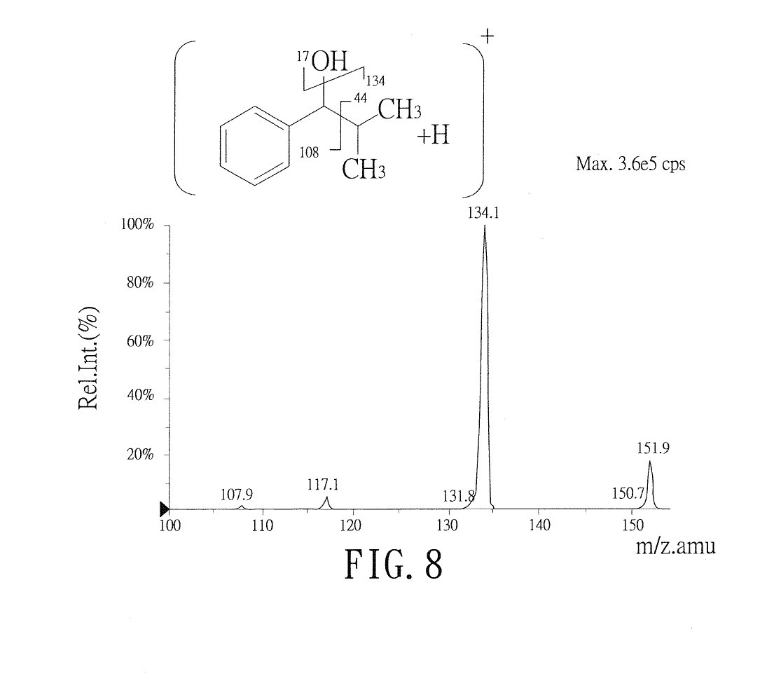

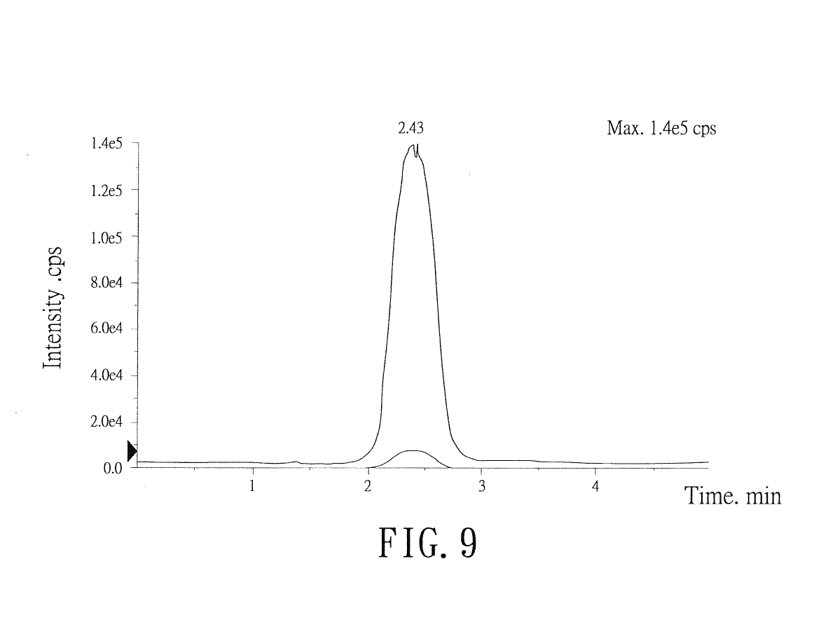

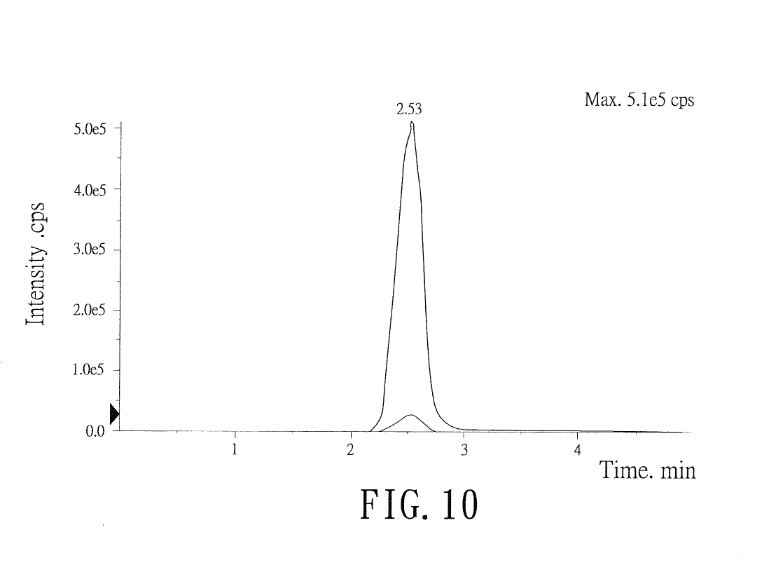

The LC/MS/MS system included two Perkin-Elmer Series 200 Micro LC pumps, a Series 200 Autosampler (Perkin-Elmer Co., Waltham, Mass.), and a AB-Sciex API-2000 triple quadrupole mass spectrometer (Applied-Biosystems, Foster City, Calif.) with TurbolonSpray probe. The data was analyzed by AB-Sciex software (Analyst version 1.3.1). The column for HPLC analysis was Polaris C-18A column (2 mm i.d.×50 mm; 3 μm particle size) (Varian Inc. Palo Alto, Calif.). After biosynthesis reaction, the supernatant was filtrated, desalted and divided into a 96-well plate. 5 μl of the sample was loaded into the LC by autosampler, and after the sample was segregated by C18 column, the segregated sample was loaded into the MS/MS. The chromatography condition was as following: the analysis time was 5 minutes, the mobile phase was a mixture solution containing H₂O (containing 0.1% formic acid) and ethanol, which the ratio of H₂O and ethanol was 70:30, and the flow rate was 100 μl/min. The MS/MS detection condition was as following: the TurbolonSpray was set on 5500 V, the orifice voltage was set on 80 V, the temperature was set on 200° C., the collision energy was set on 28 eV, and the entrance potential was set on −9 V. The collision gas (nitrogen) pressure was kept on 2.3×10⁻⁵ ton. The norephedrine was detected by positive ion mode and multiple-reaction monitoring mode. The mass to charge ratio for the analysis of the norephedrine standard was m/z 151.9-134.1 and m/z 151.9-117.1, and the retention time of the norephedrine standard was about 2.5 minutes. Please refer to FIG. 7 to FIG. 10. FIG. 7 is a diagram of ion spectra of the norephedrine standard. FIG. 8 is a diagram of ion spectra of the norephedrine standard after decomposition. FIG. 9 is a mass chromatogram of the norephedrine standard. FIG. 10 is a mass chromatogram of the norephedrine biosynthesized by E. coli (pQE-CvTA). According to the analysis result, the biosynthesis product in the present invention was consistent with the norephedrine standard. Therefore, the product was confirmed to be norephedrine.

### Embodiment 4

Biosynthesis of norephedrine by transformed E. coli co-expressing acetohydroxyacid synthase and transaminase:

The transformed E. coli containing pQE-CoTA plasmid was incubated, and the incubation and induction method was the same as Embodiment 2. In first step, the L-phenylacetylcarbinol was biosynthesized through a method catalyzed by acetohydroxyacid synthase. The condition for biosynthesizing the L-phenylacetylcarbinol and the product analysis condition was the same as Embodiment 2. The benzaldehyde was confirmed to be totally consumed by HPLC analysis. Then, the L-alanine was added as amino donor, and 1 mM phosphopyridoxal was added as coenzyme. The reaction was continuously performed at 37° C. The reaction product was analyzed by HPLC and LC/MS/MS. The HPLC analysis condition was the same as Embodiment 2. The result of HPLC analysis confirmed that the yield of the norephedrine was about 85%. The optical property of the product was analyzed by Chiral-HPLC, and the analysis condition was the same as Embodiment 2. According to the result of the Chiral-HPLC analysis, the retention time of the final product was consistent with that of the norephedrine standard. The optical property of the product was confirmed to be (1R, 2S)-norephedrine. The LC/MS/MS analysis condition was the same as Embodiment 3. The analysis result showed that the product biosynthesized by transformed strain was consistent with the norephedrine standard.

### Embodiment 5

Biosynthesis of Norephedrine by Benzylamine and Pyruvate as Starting Substrates:

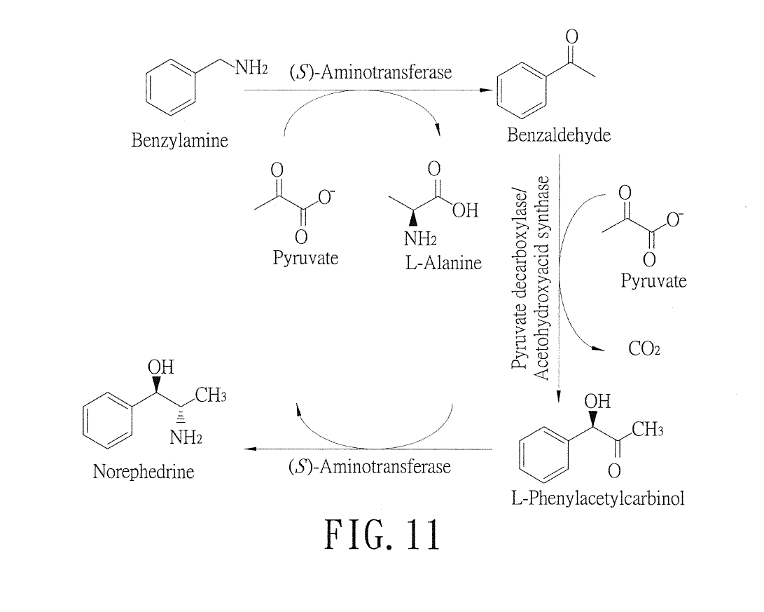

Please refer to FIG. 11. FIG. 11 is an illustration of a chemical reaction of biosynthesizing norephedrine from catalysis of transaminase (aminotransferase) and acetohydroxyacid synthase pair to the reaction between benzylamine and pyruvate. First, the transamination between benzylamine and pyruvate was catalyzed by transaminase [(S)-Aminotransferase)] to produce benzaldehyde and L-alanine. Next, the benzaldehyde was converted to L-phenylacetylcarbinol catalyzed by pyruvate decarboxylase and acetohydroxyacid synthase. Then, the transamination between the L-phenylacetylcarbinol and the L-alanine was catalyzed by transaminase [(S)-Aminotransferase)] to give the final product, norephedrine, and the byproduct, pyruvate. The pyruvate was recycled in the reaction system for the next transamination of the benzylamine and the next conversion of the benzaldehyde.

The transformed E. coli containing pQE-CoTA plasmid was incubated, and the incubation and induction method was the same as Embodiment 2. The induced bacterial broth culture was centrifuged by 6000 rpm for 15 minutes at 4° C. After centrifuging, the supernatant was discarded, and the wet weight of the bacterial cells was measure. One gram (wet weight) of the bacterial cells was suspended in a reaction solution containing 10 mM benzylamine, 20 mM pyruvate, 1 mM pyridoxal phosphate, and 100 mM sodium phosphate buffer (pH 7.0) and incubated at 37° C. After incubation, the reaction solution was collected and analyzed by HPLC and LC/MS/MS. The HPLC analysis condition was the same as Embodiment 2. According to the result of the HPLC analysis, the yield of the norephedrine was about 77%. The optical property of the final product was analyzed by Chiral-HPLC, and the Chiral-HPLC analysis condition was the same as the Embodiment 2. According to the result of the Chiral-HPLC analysis, the retention time of the final product was consistent with that of the norephedrine standard. Therefore, the optical property of the final product was confirmed to be (1R, 2S)-norephedrine. The LC/MS/MS analysis condition was the same as Embodiment 3. According to the result of LC/MS/MS analysis, the final product biosynthesized by transformed strain was consistent with the norephedrine standard.

According to the above description, in comparison with the traditional synthesis method of norephedrine, the biosynthesis method in the present invention has the advantages as following:
1. The biosynthesis process catalyzed by enzyme in the present invention is simpler. The efficiency of enzyme reaction is high so that the reaction time is shorter. The cells and enzymes can be mobilized to increase their stability and can be reused. Moreover, the reaction product has optical specificity, so the optical isomers are not separated and purified from the reaction product.
2. The major solvent of the enzyme catalysis or the whole-cell biotransformation in the present invention is water, so that the reaction condition is mild. The reaction is not performed under very low or high temperature and is not performed under special pressure to greatly decrease the dangerous of the chemical reaction and the environment pollution.
3. The formation of the byproduct in the process of the present invention is less and predictable. Furthermore, the pyruvate is regenerated in the reaction system so that the pyruvate is not added again. Therefore, the reusability of the whole-cell bioconverter is increased, the control of the reaction process is simplified, and the reaction cost is reduced.

However, the foregoing embodiments and drawings does not limits the product structures or uses of the present invention, it will be obvious to those skilled in the art that various modifications may be made without departing from the spirit and the scope of the present invention.

What is claimed is:

1. A biosynthesis method of norephedrine with specific optical activities comprising the steps of
biosynthesizing L-phenylacetylcarbinol (L-PAC); and
performing transamination reaction catalyzed by a transaminase to convert the L-phenylacetylcarbinol to the norephedrine;
thereby a two-step biosynthesis of the norephedrine is accomplished.

2. The method as claimed in claim 1, wherein the transaminase catalyzes the substitution of a carbonyl group for an amino group from an amino donor.

3. The method as claimed in claim 2, wherein the origin of the transaminase is a bacterial strain with the transaminase activity, a transformed strain with the transaminase activity, or a purified enzyme with the transaminase activity.

4. The method as claimed in claim 1, wherein the L-phenylacetylcarbinol (L-PAC) biosynthsis is catalyzed by pyruvate decarboxylase or acetohydroxyacid synthase.

5. The method as claimed in claim 4, wherein the origin of the pyruvate decarboxylase is a bacterial strain with the pyruvate decarboxylase activity, a transformed strain with the pyruvate decarboxylase activity, or a purified enzyme with the pyruvate decarboxylase activity.

6. The method as claimed in claim 4, wherein the origin of the acetohydroxyacid synthase is a bacterial strain with the acetohydroxyacid synthase activity, a transformed strain with the acetohydroxyacid synthase activity, or a purified enzyme with the acetohydroxyacid synthase activity.

7. A biosynthesis method of norephedrine with specific optical activities comprising the steps of
performing a transamination reaction between benzylamine and pyruvate catalyzed by a transaminase to produce benzaldehyde; and L-alanine;
producing L-phenylacetylacrbinol (L-PAC) from the benzaldehyde catalyzed by pyruvate decarboxylase and acetohydroxyacid synthase; and
performing a transamination reaction between the L-phenylacetylacrbinol and the L-alanine catalyzed by the transaminase to produce final product, norephedrine, and byproduct, pyruvate.

8. The method as claimed in claim 7, wherein the origin of the pyruvate decarboxylase is a bacterial strain with the pyruvate decarboxylase activity, a transformed strain with the pyruvate decarboxylase activity, or a purified enzyme with the pyruvate decarboxylase activity.

9. The method as claimed in claim 7, wherein the origin of the acetohydroxyacid synthase is a bacterial strain with the acetohydroxyacid synthase activity, a transformed strain with the acetohydroxyacid synthase activity, or a purified enzyme with the acetohydroxyacid synthase activity.

10. The method as claimed in claim 7, wherein the transaminase catalyzes the substitution of a carbonyl group for an amino group from an amino donor.

11. The method as claimed in claim 10, wherein the origin of the transaminase is a bacterial strain with the transaminase activity, a transformed strain with the transaminase activity, or a purified enzyme with the transaminase activity.
